(19) United States
(12) Patent Application Publication
(10) Pub. No.: US 2010/0271213 A1
(43) Pub. Date: Oct. 28, 2010

(54) Process and Device for Determining Recommendations for Active Ingredient Dosages on the Basis of Series of Measurements of at Least One Physiological Parameter of a Patient

(76) Inventors: Michael Krainz, Graz (AT); Marco Jean Pierre Leiner, Graz (AT)

Correspondence Address: BAKER & DANIELS LLP / ROCHE, 300 NORTH MERIDIAN STREET, SUITE 2700, INDIANAPOLIS, IN, 46204, US

(21) Appl. No.: 12/766,455

(22) Filed: Apr. 23, 2010

(30) Foreign Application Priority Data
Apr. 23, 2009 (EP) 09450088.1

(51) Int. Cl. G08B 23/00; G06F 19/00; G01N 33/50

(52) U.S. Cl. 340/573.1; 702/19

(57) ABSTRACT

A process comprises performing a series of measurements of at least one physiological parameter, in particular a blood value, such as, e.g., blood sugar, on a patient, wherein samples are taken from the patient at discrete measuring times, with the measurements being performed on those samples. A recommendation for a dosage of an active ingredient to be administered to the patient until the next measuring time is determined from the measurements, for which purpose a dosage proposal algorithm incorporating the at least one measured physiological parameter is applied. As a result, it is possible to adjust at least one physiological parameter of the patient to a target range or keep it in the target range, respectively. The dosage proposal algorithm is configured such that the next measuring time is determined in consideration of measurement exclusion time windows and is optionally reported to the person in charge.

### PRIORITY

This application claims the benefit of European Patent Application 0945008.1 filed on Apr. 23, 2009, the disclosure of which is incorporated herein by reference.

### FIELD

The present disclosure relates to a process for carrying out a series of measurements of at least one physiological parameter. More particularly, the present disclosure relates to a process for carrying out a series of measurements of a blood value, such as, e.g., blood sugar, on samples taken from a patient at discrete measuring times and for calculating a recommendation for a dosage of at least one active ingredient to be administered to the patient until the next measuring time on the basis of a dosage proposal algorithm incorporating the at least one measured physiological parameter, whereby at least one physiological parameter of the patient is adjusted to a target range or kept in said target range, respectively. Furthermore, the disclosure relates to a medical-diagnostic analyzer for carrying out the above process.

### BACKGROUND AND SUMMARY

In many medical treatments of patients it is necessary to regularly monitor certain physiological parameters of the patient and to bring them to a defined range of values or keep them in said range of values, respectively, by administering active ingredients. For example, hyperglycemia (i.e., excessively high blood sugar levels (above 110 mg/dl)) may occur postoperatively in patients, also in non-diabetics, in the intensive care unit. A normalization of the blood sugar level by continuous glucose measurement in connection with a selective insulin administration (Tight Glycemic Control) in said phase results in a significant decrease in the mortality rate. This correlation was for the first time mentioned in a study in 2001 and has since then been confirmed several times. Computer-implemented algorithms have already been developed which assist the hospital staff in dosing the insulin administration. Such an algorithm, which has proved its worth in practice, was developed as a “Glucommander”-algorithm by researchers of Atlanta Diabetes Associates and is described, e.g., in the article “Intravenous Insulin Infusion Therapy; Indications, Methods, and Transition to Subcutaneous Insulin Therapy”, Bode et al, ENDOCRINE PRACTICE, Vol 10 (Suppl 2) March/April 2004 as well as in an article by Davidson et al. in Diabetes Care, Vol. 20, No. 10, 2418-2423, 2005. The principles of the “Glucommander”-algorithm can be illustrated in a Cartesian coordinate system with the blood sugar level as the abscissa and an insulin dose [units per hour] as a pencil of lines, with each straight line representing a different multiplier. Vertical lines in said diagram define a blood sugar range to which the patient is to be brought or in which he or she is to be kept, respectively. The multiplier lines indicate how fast the change in the blood sugar level should occur, in other words, how high the insulin dose chosen should be until the next measuring time. After every new measurement of the blood sugar level, the active ingredient dose to be administered is re-evaluated, and the multiplier can be exchanged. The measurement of the blood sugar level may be performed with a commercially available blood glucose measuring device, for example, a special blood glucose measuring device or also a multiparameter measuring device such as, for example, a blood gas analyzer for determining blood gases, electrolytes and metabolites (glucose, lactate).

The “Glucommander”-algorithm has proved to be valuable for assisting the nursing staff in intensive care. However, a precondition for its successful application is that the prescribed intervals between two blood sugar level measurements are observed precisely. This, however, cannot be guaranteed for various reasons, but, in practice, measurement exclusion time windows exist in which no measurements are possible. Such measurement exclusion time windows may be caused by the measuring device, for example, if the measuring device has to undergo a periodic calibration or other internal maintenance and test procedures. Measurement exclusion time windows may also be caused by the user, for example, if a patient is not available for a blood sugar level measurement for some time since he or she has to handle different examinations or performances. If measurement exclusion time windows coincide with prescribed times of blood sugar level measurements, the recommendation of the “Glucommander”-algorithm will be suboptimal. The potential for an occurrence of hyper- or hypoglycemias is increased if the blood sugar values of a patient can no longer be controlled, especially in the postoperative area.

Accordingly, physiological parameters of a patient to be monitored have to be collected in a series of measurements in order to calculate a recommendation for a dosage of an active ingredient to be administered to the patient until the next measuring time on the basis of said series of measurements using a dosage proposal algorithm, wherein the recommended dosage may not increase the health risk for the patient even if the intended measuring times lie in measurement exclusion time windows. In particular, the present disclosure provides for avoiding blood sugar levels that can no longer be controlled with the recommendation values which have been determined as described above, thus increasing the risk of the occurrence of hyper- or hypoglycemias, if the recommended measuring times cannot be observed.

The present disclosure provides a process for carrying out a series of measurements of at least one physiological parameter, in particular a blood value, such as, e.g., blood sugar, on samples (S) taken from a patient at discrete measuring times (n) and for calculating a recommendation (DS) for a dosage of at least one active ingredient to be administered to the patient until the next measuring time on the basis of a dosage proposal algorithm incorporating the at least one measured physiological parameter, whereby at least one physiological parameter of the patient is adjusted to a target range, characterized in that the next measuring time (n+1, (n+1)′, (n+1)″) is determined in consideration of measurement exclusion time windows (EX).

The present disclosure further provides a medical-diagnostic analyzer such as, for example, a blood analyzer, for receiving samples (S) taken from a patient at discrete measuring times, comprising at least one measuring sensor (3) for measuring at least one physiological parameter, in particular a blood value, such as, e.g., blood sugar, on the samples, an arithmetic unit (4) receiving the measuring signals (MS) of the at least one measuring sensor for processing measured values of the at least one physiological parameter from the measuring signals, for calculating a recommendation (DS) for a dosage of an active ingredient to be administered to the patient until the next measuring time on the basis of a dosage proposal algorithm incorporating the at least one measured physiological parameter, wherein the arithmetic unit (4).

The process according to the disclosure comprises performing a series of measurements of at least one physiological parameter, in particular a blood value, such as, e.g., blood sugar, of a patient, wherein samples are taken from the patient at discrete measuring times, with the measurements being performed on those samples. A recommendation for a dosage of at least one active ingredient to be administered to the patient until the next measuring time is determined from the measurements, for which purpose a dosage proposal algorithm incorporating the at least one measured physiological parameter is applied. As a result, it is possible to adjust at least one physiological parameter of the patient to a target range or keep it in the target range, respectively. It should be mentioned that the at least one physiological parameter of the patient which is adjusted to a target range or kept therein, respectively, is not necessarily the physiological parameter which is measured in the samples. In fact, it is also within the scope of the present disclosure to perform indirect measurements, i.e., to measure a physiological parameter which is associated with the physiological parameter to be adjusted and, during the administration of the active ingredient, changes in a way associated with the physiological parameter to be adjusted. The dosage proposal algorithm is configured such that the next measuring time is determined in consideration of predetermined measurement exclusion time windows and reported to the person in charge. In an embodiment of the disclosure, the determined next measuring time represents a variable of the dosage proposal algorithm, i.e., the proposed active ingredient dose is calculated in consideration of altered time intervals between the measurements and/or by recalculating the value of the at least one physiological parameter which is to be expected in the next measurement.

The term “active ingredient” is to be understood as comprising also any pharmaceutical preparation containing said active ingredient.

It is also within the scope of the disclosure that a plurality of physiological parameters are measured, which are utilized by the dosage proposal algorithm for calculating the dosage recommendation for an active ingredient. It is known per se to use several physiological parameters for calculating a dosage and for recommending the dose of an active ingredient, respectively, see, e.g., US 2007/0168136 A.

In another embodiment of the disclosure, the dosage proposal algorithm includes patient data entered by a user, such as weight, food habits etc., in the calculation of the recommendation. This measure is known per se, see, e.g., WO 2008 057213, in which it is disclosed that several physiological parameters (e.g., weight, body temperature) are used for calculating a dosage of an active ingredient (insulin). In doing so, a distinction must be made between known preset parameters, such as the weight of the patient, and physiological parameters which are measured continuously. Both groups of physiological parameters can be used by the dosage proposal algorithm for calculating a recommendation for an active ingredient administration.

In one embodiment of the disclosure, the next measuring time is determined by adding a time interval to the latest measuring time and checking whether the preliminary next measuring time resulting therefrom lies in a measurement exclusion time window and, if applicable, the next measuring time is shifted outside of the measurement exclusion time window. In this embodiment, it is not necessary to consider whether the measurement exclusion time window occurs caused by the analyzer or caused by the user. In order to rule out a possible undesired situation for the patient, it is envisaged that the shifting of the next measuring time outside of the measurement exclusion time window occurs by precalculating a measured value to be expected and by assessing the liklihood of whether the measured value to be expected is acceptable for the condition of the patient. Alternatively, the shifting of the next measuring time outside of the measurement exclusion time window can occur while the determination of a maximum admissible time span between two measuring times for the condition of the patient is being assessed. Should an undesired result come from the above-mentioned risk assessments, an alert is given to the user, wherein the measuring time is optionally shifted before the measurement exclusion time window.

If the measurement exclusion time window is caused by the measuring device, in one embodiment of the disclosure, it is envisaged that the measurement exclusion time window caused by the measuring device is shifted outside of the next measuring time, if the preliminary next measuring time which has been calculated lies in the measurement exclusion time window. Said embodiment provides that the measurements and dosage recommendations can be continued as planned.

If the measurement exclusion time window is caused by the user, in one embodiment of the disclosure it is envisaged that, in case the preliminary next measuring time which has been calculated lies in the measurement exclusion time window, the user is recommended to shift his or her actions leading to the measurement exclusion time window such that they will not collide with the next measuring times which have been calculated. Thereupon, the user can shift the measurement exclusion window caused by the user outside of the next measuring time and, optionally, can shift also subsequent measurement exclusion windows which are caused by the user. Said embodiment provides the advantage that the measurements and dosage recommendations can be continued as originally planned.

A medical-diagnostic analyzer for carrying out the process according to the disclosure which is designed, for example, as a blood analyzer, comprises at least one measuring sensor for measuring at least one physiological parameter on the samples taken from a patient at discrete measuring times. In one embodiment of the analyzer, at least one sample receiver for receiving the samples taken from a patient at discrete measuring times is provided, with the at least one measuring sensor for measuring the at least one physiological parameter on the samples communicating with the at least one sample receiver. The measuring signals of the at least one measuring sensor are received by an arithmetic unit and processed from the measuring signals into measured values of the at least one physiological parameter. The measured values are used in a dosage proposal algorithm incorporating the at least one physiological parameter for calculating a recommendation for a dosage of at least one active ingredient to be administered to the patient until the next measuring time. This recommendation as well as alerts and other messages are given by the arithmetic unit to a user via an output interface. Preferably, the arithmetic unit is designed for performing the process in parallel for a plurality of patients.

In summary, the disclosure provides the following: The next measuring time is determined such that the measuring device is safely ready to measure. The level of the active ingredient dose to be administered is adjusted according to the shifting of the measuring time. Due to the calculation of the value of the physiological parameter to be expected, early responses to various actions are possible. Based on the knowledge about device and/or user actions, the next measuring time and the recommendation for the active ingredient dosage can be optimized.

### BRIEF DESCRIPTION OF THE DRAWINGS

The disclosure is now illustrated in further detail by way of exemplary embodiments, with reference to the drawings in which:

FIG. 1 shows a diagram of a medical-diagnostic analyzer by means of which the process according to the disclosure is carried out;

FIGS. 2 and 3 show schematic time charts for illustrating embodiments of the process according to the disclosure; and

FIG. 4 shows blood sugar level developments of a patient over time as a function of dosage recommendations.

### DETAILED DESCRIPTION OF THE DRAWINGS

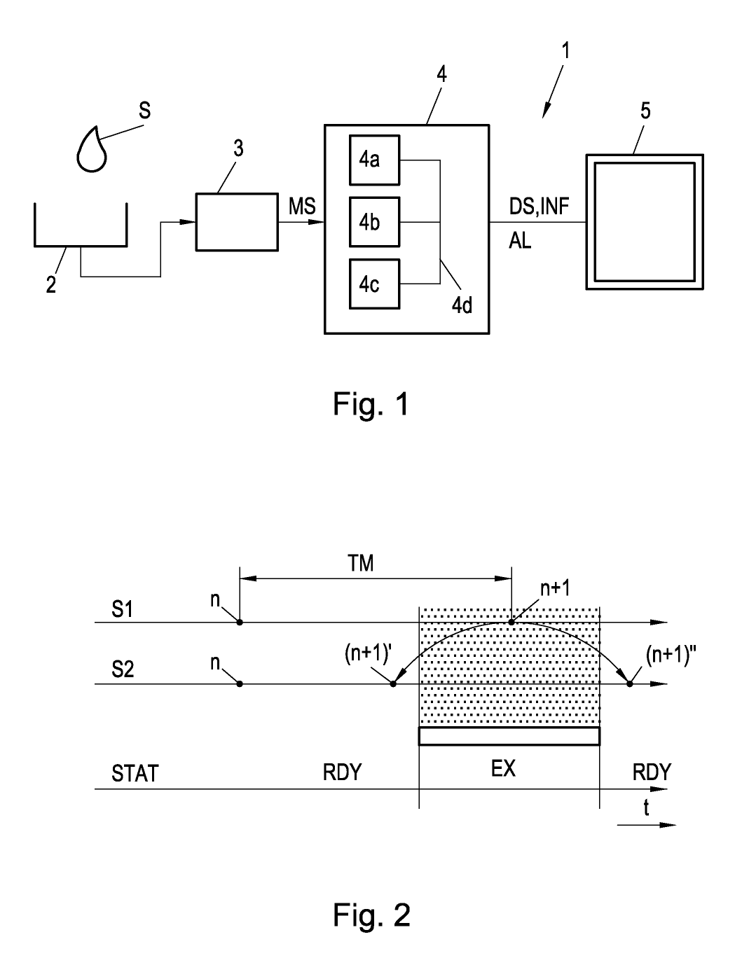

In FIG. 1, a medical-diagnostic analyzer 1 is schematically illustrated in a block diagram. Said medical-diagnostic analyzer 1 comprises a sample receiver 2 for receiving samples S taken from a patient at discrete measuring times, at least one measuring sensor 3 communicating with the sample receiver for measuring at least one physiological parameter, in particular a blood value, such as, e.g., blood sugar, on the samples S, furthermore, an arithmetic unit 4 receiving the measuring signals MS of the at least one measuring sensor 3 for processing measured values of the physiological parameters from the measuring signals MS. From the measured values, the arithmetic unit 4 calculates a recommendation for a dosage of at least one active ingredient to be administered to the patient until the next measuring time on the basis of a dosage proposal algorithm incorporating the at least one physiological parameter which has been measured. The arithmetic unit 4 comprises at least one processor 4a, a program memory 4b and a main memory 4c which are interconnected by a bus system 4d. As it has been described so far, the analyzer 1 can be constructed on the basis of a commercially available blood gas analyzer for determining blood gases, electrolytes and metabolites (glucose, lactate) or of another blood glucose measuring device, which are produced and marketed by the applicant.

The medical-diagnostic analyzer differs from known analyzers by a workflow implemented therein for performing series of measurements and a process (algorithm) for calculating a recommendation for an active ingredient dose to be administered between two measuring times. Within the series of measurements, the relevant physiological parameters are determined discretely over time by manual sampling and measurement. Alternatively, automated sampling and/or measuring steps are also possible. The dosage proposal algorithm is implemented as an executable program which is stored in the program memory 4b and processed by the arithmetic unit 4. The result of the calculations of the arithmetic unit 4 is a dosage recommendation DS for a user of the analyzer 1 for a continuous or periodic delivery or a delivery following another administration profile of at least one active ingredient to a patient (e.g. by infusions). However, alternatively or additionally, it may comprise alerts AL and general messages INF. The dosage recommendation DS, alerts AL and messages INF are transmitted by the arithmetic unit 4 to an output interface 5 which is implemented, for example, as a display, printer, etc. The analyzer 1 is designed such that the arithmetic unit 4 performs series of measurements in parallel for a plurality of patients and calculates active ingredient dosage recommendations.

It should be noted that, in this type of medical-diagnostic analyzer 1, there are often measurement exclusion time windows caused by the measuring device, for which device actions such as, e.g., a system calibration have to take place and for which consequently no measurements can be carried out. Furthermore, measurement exclusion time windows caused by the user may exist, for example, because of work-related circumstances which likewise prevent a measurement.

The medical-diagnostic analyzer 1 functions such that the measurement exclusion time windows are considered in the implemented dosage proposal algorithm, with the determined next measuring time preferably representing a variable of the dosage proposal algorithm. As illustrated in the time chart of FIG. 2, in a first step S1, the next measuring time n+1 is determined by adding a time interval TM to the latest measuring time n and subsequently checking whether the preliminary next measuring time n+1 resulting thereform lies in a measurement exclusion time window EX. This is the case here and therefore, in a step S2, the next measuring time is shifted outside of the measurement exclusion time window EX and into a time period RDY in which the analyzer 1 is operable, as can be seen in the status line STAT in FIG. 2. The shifting of the next measuring time can be shifted either before (n+1)′ the measurement exclusion time window EX or behind it (n+1)″. With the shifting of the next measuring time, optionally, a corresponding adjustment of the active ingredient dose will be carried out as well. The decision whether the next measuring time should be shifted before (n+1)′ or behind (n+1)′ the measurement exclusion time window EX can be made according to the following case differentiations:

Case 1: The calculated next measuring time n+1 lies in the first half of the measurement exclusion time window EX. Then, the measuring time (n+1)′ is shifted before the beginning of the measurement exclusion time window EX.

If the dosage recommendation algorithm used is configured such that a particular time interval TMmin (e.g., 15 min) between consecutive measurements should not be fallen short of in order to obtain reliable dosage recommendations for an active ingredient administration, the following special case arrangements can be differentiated:

Case 1a: The calculated next measuring time n+1 lies in the first half of the measurement exclusion time window EX and the time distance between the time of the current measurement n and a measuring time (n+1)′ to be shifted before the beginning of the measurement exclusion time window EX according to the above assumption is, optionally in consideration of the duration of carrying out a measuring process, smaller than the time interval TMmin not to be fallen short of In order to avoid falling short of the time interval TMmin not to be fallen short of, the next measuring time (n+1)″ is shifted in this case after the end of the measurement exclusion time window EX.

Case 1b: The calculated next measuring time n+1 lies in the first half of the measurement exclusion time window EX and the time distance between the time of the current measurement n and a measuring time (n+1)′ to be shifted before the beginning of the measurement exclusion time window EX according to the above assumption is, optionally in consideration of the duration of carrying out a measuring process, larger than the time interval TMmin not to be fallen short of In this case, the next measuring time (n+1)′ is shifted before the beginning of the measurement exclusion time window EX.

Case 2: The calculated next measuring time n+1 lies in the second half or precisely in the half time of the measurement exclusion time window EX. Then, the measuring time (n+1)″ is shifted after the end of the measurement exclusion time window EX.

If the dosage recommendation algorithm used is configured such that a particular time interval TMmax (e.g., 60 min) between consecutive measurements should not be exceeded in order to obtain reliable dosage recommendations for an active ingredient administration, the following special case arrangements can be differentiated:

Case 2a: The calculated next measuring time n+1 lies in the second half or precisely in the half time of the measuring time exclusion window EX and the time distance between the time of the current measurement n and a measuring time (n+1)″ to be shifted after the beginning of the measurement exclusion time window EX according to the above assumption is, optionally in consideration of the duration of carrying out a measuring process, larger than the time interval TMmax not to be exceeded. So as not to exceed the time interval TMmax not to be exceeded, the next measuring time (n+1)′ is shifted in this case before the beginning of the measurement exclusion time window EX.

Case 2b: The calculated next measuring time n+1 lies in the second half or precisely in the half time of the measuring time exclusion window EX and the time distance between the time of the current measurement n and a measuring time (n+1)″ to be shifted after the beginning of the measurement exclusion time window EX according to the above assumption is, optionally in consideration of the duration of carrying out a measuring process, smaller than the time interval TMmax not to be exceeded. In this case, the next measuring time (n+1)″ is shifted after the end of the measurement exclusion time window EX.

Such minimum or maximum time intervals between consecutive measuring times may become relevant particularly if it should be guaranteed that consecutive measuring times are spaced apart as regularly as possible in order to enable an adjustment of the patient to a particular target value of a physiological parameter, e.g., of the blood sugar value, which is as ideal as possible.

For calculating the possible shifting of the measuring time, such a possible dosage recommendation algorithm includes the following aspects:

The distance between the latest measurement (n) and a measurement (n+1)″ shifted backward ensures an acceptable risk.

The precalculated measured value of the physiological parameter which is to be expected ensures an acceptable risk.

If the next measuring time is shifted forward or backward, the expected measured value of the subsequent measurement of the physiological parameter can be precalculated and, depending thereupon, the recommended active ingredient dose can be altered, in case the precalculated measured value does not ensure an acceptable risk, as will be explained below.

FIG. 4 shows an exemplary diagram of a blood sugar level BG of a patient over time t. At measuring time n, the dosage proposal algorithm creates a dosage recommendation DS for the delivery of an active ingredient (in this example insulin) to the patient. The dosage recommendation DS is dimensioned such that the course of the blood sugar level BG(DS) should reach a desired blood sugar level BGS at the time n+1 of the next measurement. If it is shifts the time of the next measurement forward (n+1)′ or backward (n+1)″ due to measurement exclusion time windows, an upward deviation D′ from the desired blood sugar level BGS will occur at the measuring time (n+1)′ shifted forward and a downward deviation D″ will occur at the measuring time (n+1)″ shifted backward, respectively. On the one hand, the dosage proposal algorithm can now allow for these deviations at the measuring time (n+1)′, (n+1)″ by means of interpolation by taking into account the difference values as the desired value of the blood sugar level and taking these deviating values as a basis when a new dosage recommendation is calculated. Furthermore, it performs a risk assessment to find out whether the deviations, especially with measuring times shifted backward, are possibly so large that complications for the patient are to be taken into account and thus the shifting of the measuring time is unacceptable and the user has to be warned. However, the dosage proposal algorithm can also allow for the altered measuring times (n+1)′, (n+1)″ insofar as it delivers altered dosage recommendations DS′, DS″ which result in blood sugar level developments BG(DS′), BG(DS″) in the patient which allow the desired blood sugar level BGS to be achieved at the altered measuring times (n+1)′, (n+1)″.

On the basis of example cases, further variants of embodiments of the disclosure are now illustrated, wherein a time for the next measurement n+1 is calculated in a step S1 and it is then checked whether the calculated next measuring time n+1 lies in a measurement exclusion time window EX in which no measurement can be carried out either caused by the device or caused by the user.

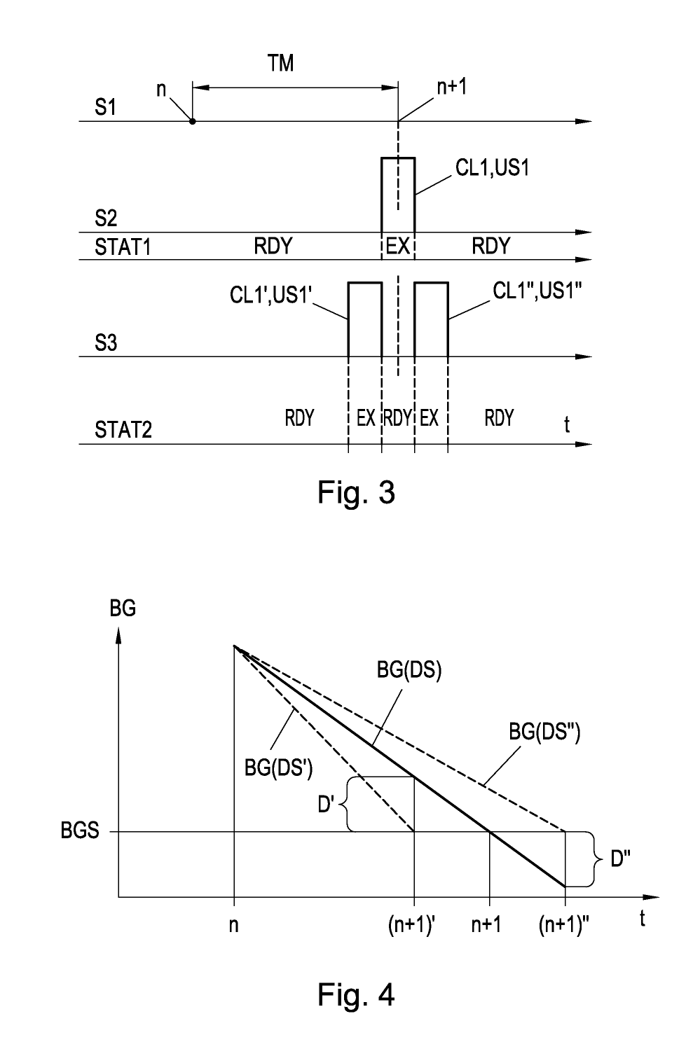

FIG. 3 shows a time chart in which measurement exclusion time windows occur caused by actions CL1 of the analyzer 1, e.g., caused by internal calibration processes, or caused by actions US1 of a user. As can be seen from line S2 and status line STAT1, the actions CL1, US1 of the analyzer 1 or of the user, respectively, would coincide with the time n+1 of the next measurement calculated from the time interval TM from the previous measuring time n, i.e., would define a measurement exclusion time window EX which includes the measuring time n+1. In order to avoid this, the planned actions CL1 and US1, respectively, are shifted in a step S3, namely either forward (CL1′, US1′) or backward (CL1″, US1″). This occurs in consideration of the fact that an acceptable risk is ensured. As can be seen from the status line STAT2, the measurement exclusion time windows EX resulting from the shifting of the actions of the analyzer thus occur during times which do not coincide with the planned time n+1 of the next measurement. This means that the analyzer 1 is ready to measure (RDY) at the planned measuring time n+1.

A further embodiment of the disclosure concerns the case in which the measurement exclusion time window is too large for performing a shifting of the next measuring time without risk. In this case, the algorithm shifts the time of the next measurement before the measurement exclusion time window and outputs an alert AL to the user indicating that it is not guaranteed that the target values of the physiological parameter will be reached or maintained and that the user has to take separate measures.

Hereinafter, the procedure of the process according to the disclosure for the application example of monitoring blood values, in particular the blood sugar level, of patients using the above-illustrated analyzer 1 is described. In this use case, the blood sugar values of a plurality of patients are monitored in parallel in the point of care area (in particular in the intensive care unit) by a separate series of measurements per each patient, using manual blood sugar measurements. After each blood sugar measurement, an insulin dose to be administered until the next measuring time is recommended for each patient via an appropriate algorithm. An increased glucose level shall be lowered by an insulin dose steadily administered to the patient by means of a dosing pump and stabilized within a defined target range. The time of the next measurement is determined and displayed together with the insulin dose. Furthermore, a silent alert (display) is to be triggered when said time is reached.

In the adjustments of the analyzer 1, the following can be adjusted globally (i.e., uniformly for all patients): the maximum interval between two measurements within a series of measurements and the maximum value of the insulin dose to be calculated

Furthermore, patient data such as date of birth, weight, food habits, insulin sensitivity factors etc., which the dosage proposal algorithm should include in the calculation of the recommendation (DS) for the insulin dosage, can be adjusted individually (i.e., separately for each patient) in the adjustments of the analyzer 1.

A series of measurements is started during the measurement after the patient identification (ID) has been entered. The starting behaviour of the dosage proposal algorithm (mild, normal, user-defined), a starting multiplier (0.5 to 2.0) and a glucose target value (or target range) can be determined individually for each patient in the first measurement.

The withdrawal of blood (venously or arterially) is done manually with conventional sampling vessels (syringes or the like). The sampling vessel is contacted manually with the analyzer 1 and the measurement is started, whereupon at least one aliquot of the sample is sucked automatically into the analyzer 1 and whereupon the measurement takes place. As soon as the glucose value is measured on the analyzer 1, the implemented algorithm calculates the needed insulin dose on the basis of the measured glucose value, which insulin dose is to be steadily administered to the patient until the next measuring time using a dosing pump. The time of the next measurement is displayed together with the insulin dose.

The data are stored in the database of the analyzer 1 and optionally transferred to a LIS/HIS (hospital information system).

The user doses the insulin administration on the patient using a dosing pump and has to confirm the insulin dose which has actually been administered on the analyzer not later than at the beginning of the subsequent measurement of the same series of measurements.

Optionally, the user can administer an altered insulin dose to the patient and has to confirm said dose on the analyzer 1 along with a comment.

The imminent measurements of all active series of measurements are managed in an alert list, and the analyzer shows the user by means of a silent alert that a measurement is to be performed.

Optionally, the user can invoke a trend chart of the current patient during the measurement. The trend chart displays the measured glucose value and the insulin dose which has actually been administered for the entire duration of the series of measurements or a part of this duration. Furthermore, the user can invoke a trend chart for any patient in the database.

The dosage proposal algorithm implemented in the analyzer 1 is, for example, an advanced development according to the disclosure of the “Glucommander”-algorithm, as described in the initially mentioned document “Intravenous Insulin Infusion Therapy; Indications, Methods, and Transition to Subcutaneous Insulin Therapy”, Bode et al, ENDOCRINE PRACTICE, Vol 10 (Suppl 2) March/April 2004.

The “Glucommander”-algorithm is based on the formula:

IR(k)=MM(k)×(BG(k)−TH)

with:
IR=insulin dose [units per hour]
k=iteration step [equivalent to measuring times n, n+1, . . . ]
MM=multiplier
BG=blood sugar level of the patient, and
TH=minimum blood sugar threshold from which an administration of insulin takes place, typically determined to be 60 mg/dl.

The multiplier MM is redetermined in every iteration step. An initial value of the multiplier MM for the first measurement is usually adjusted to 0.02. For subsequent measurements, the physician can multiply the multiplier by an “aggressiveness factor” which codefines the insulin dose. Typical values of this “aggressiveness factor” are 0.5 in the mild state, 1 in the normal state or 0.5 to 2 in the variable state. The iteration steps k corresponding to the interval TM between two measuring times n, n+1 (see FIG. 2) are first determined to be 30 minutes. According to the “Glucommander”-algorithm, the multiplier MM is readjusted every hour by 0.01 in order to reach the desired blood sugar level. If the result is that the desired blood sugar level is fallen short of, a reduction by 0.01 occurs; if the result is that the desired blood sugar level is reached or maintained, no change occurs; if the result is above the desired blood sugar level and the blood sugar level has not decreased by 25%, an increase by 0.01 occurs. Details of the “Glucommander”-algorithm can be taken in particular from Appendix 3 of the quoted article by Bode et al.

The “Glucommander”-algorithm provides that the distances between the iteration steps k be observed precisely. As long as this is possible, the dosage proposal algorithm functions according to the “Glucommander”-algorithm, with the dosage recommendation DS corresponding to the insulin dose IR in the above formula. As explained above, it is not always possible, either caused by the device or caused by the user, to precisely observe the distances between the iteration steps k, i.e., measurement exclusion time windows EX exist. The present disclosure provides for moving the measuring times outside of the measurement exclusion time windows EX (FIG. 2) or shifting the measurement exclusion times EX (FIG. 3). In the present exemplary implementation of the dosage proposal algorithm, this is handled as follows:

If the preliminary next measuring time n+1 lies within the measurement exclusion time window EX, the next measuring time is shifted forward (n+1)′ or backward (n+1)″, as has been explained above on the basis of FIG. 2, in such a way that the measuring time is apart from the beginning or the end, respectively, of the time exclusion window EX by a certain time interval, for example, 5 minutes.

If, during the forward shifting of the measuring time (n+1)′, a minimum time interval TM, for example, of less than 15 minutes, is fallen short of, the risk assessment of the dosage proposal algorithm interprets said time span as too short for being able to make a reliable statement about the change in the blood sugar level of the patient during the next measurement. In this case, the next measuring time (n+1)″ is shifted to five minutes after the measurement exclusion time window EX and an alert information (AL) is delivered to the physician.

For illustrating a further implemented risk assessment, reference is again made to FIG. 4. As is evident, the (linear) blood sugar development BG(DS) would result in the desired blood sugar level BGS being fallen short of by the difference D″ during a backward shifting of the next measuring time (n+1)″. This involves the risk of hypoglycemia for the patient. Therefore, the dosage proposal algorithm performs a recalculation of an adapted recommendation DS″ if it detects an impending drop below the desired blood sugar level BGS, wherein the prolonged time interval between the measuring time n and the next measuring time (n+1)″ is used as a basis. The adapted recommendation DS″ of the insulin administration can be calculated by linear interpolation the result of which is the linear blood sugar level development BG(DS″).

The foregoing description of the invention is illustrative only, and is not intended to limit the scope of the invention to the precise terms set forth. Although the invention has been described in detail with reference to certain illustrative embodiments, variations and modifications exist within the scope and spirit of the invention as described and defined in the following claims.

1. A process for carrying out a series of measurements of at least one physiological parameter, including a blood sugar value, on samples taken from a patient at discrete measuring times and for calculating a recommendation for a dosage of at least one active ingredient to be administered to the patient until the next measuring time on the basis of a dosage proposal algorithm incorporating the at least one measured physiological parameter, whereby at least one physiological parameter of the patient is adjusted to a target range, the next measuring time being determined by considering measurement exclusion time windows.

2. The process according to claim 1, wherein the dosage proposal algorithm includes patient data, such as weight, food habits etc., in the calculation of the recommendation.

3. The process according to claim 2, wherein the determined next measuring time represents a variable of the dosage proposal algorithm.

4. The process according to claim 3, wherein the next measuring time is determined by adding a time interval to the latest measuring time and checking whether the preliminary next measuring time resulting therefrom lies in a measurement exclusion time window and, if applicable, the next measuring time is shifted outside of the measurement exclusion time window.

5. The process according to claim 4, wherein the shifting of the next measuring time outside of the measurement exclusion time window is carried out subject to precalculating a measured value to be expected, in particular by interpolation, and by assessing the risk whether the measured value to be expected is acceptable for the condition of the patient.

6. The process according to claim 5, wherein assessing the risk results in a finding of an unduly high risk, an alert information is output.

7. The process according to claim 6, wherein the measuring time is shifted to before the measurement exclusion time window.

8. The process according to claim 4, wherein the shifting of the next measuring time outside of the measurement exclusion time window is carried out considering the risk of a maximum admissible time span between two measuring times for the condition of the patient is being assessed.

9. The process according to claim 8, wherein assessing the risk results in a finding of an unduly high risk, an alert information is output.

10. The process according to claim 9, wherein the measuring time is shifted to before the measurement exclusion time window.

11. The process according to claim 1, wherein the measurement exclusion time window is caused by actions of the analyzer, the next measuring time is determined by adding a time interval to the latest measuring time and checking whether the next measuring time resulting therefrom lies within the measurement exclusion time window and the measurement exclusion time window caused by the measuring device is shifted outside of the next measuring time.

12. The process according to claim 1, wherein the measurement exclusion time window is caused by actions of the user, the next measuring time is determined by adding a time interval to the latest measuring time and checking whether the next measuring time resulting therefrom lies within the measurement exclusion time window and a meassage is transmitted to the user in which he or she is asked to shift the time of his or her actions and hence the measurement exclusion window caused by the user outside of the next measuring time.

13. A process according to claim 12, wherein the user shifts the measurement exclusion window caused by the user outside of the next measuring time and shifts subsequent measurement exclusion windows which are caused by the user.

14. A medical-diagnostic blood analyzer for receiving samples taken from a patient at discrete measuring times, comprising:
at least one measuring sensor for measuring at least one physiological blood parameter of the samples,
an arithmetic unit receiving measuring signals of the at least one measuring sensor for processing measured values of the at least one physiological blood parameter from the measuring signals, for calculating a recommendation for a dosage of an active ingredient to be administered to the patient until the next measuring time on the basis of a dosage proposal algorithm incorporating the at least one measured physiological parameter, wherein the arithmetic unit includes instructions therein causing the analyzer to:
calculate a recommendation for a dosage of at least one active ingredient to be administered to the patient until a next measuring time on the basis of a dosage proposal algorithm incorporating the at least one measured physiological blood parameter, whereby at least one physiological blood parameter of the patient is adjusted to a target range, and
determining the next measuring time by considering measurement exclusion time windows.

15. The analyzer according to claim 14, further including an output interface by which the arithmetic unit outputs the dosage recommendation, alerts, and other messages to a user.

16. The analyzer according to claim 14, wherein the arithmetic unit performs the calculating and determining steps for a plurality of patients in parallel.

17. A medical-diagnostic blood analyzer, comprising:
at least one measuring sensor for measuring at least one physiological blood parameter of the samples,
an arithmetic unit including instructions therein causing the analyzer to:
receive measuring signals of the at least one measuring sensor taken from a patient at discrete measuring times,
process the measuring signals to determine measured values of the at least one physiological blood parameter;
calculate a recommendation for a dosage of an active ingredient to be administered to the patient until the next measuring time on the basis of a dosage proposal algorithm incorporating the at least one measured physiological blood parameter, whereby at least one physiological parameter of the patient is adjusted to a target range, and
determining the next measuring time by considering measurement exclusion time windows.

18. The analyzer of claim 17, further comprising an output interface by which the arithmetic unit outputs the dosage recommendation, alerts, and other messages to a user.

19. The analyzer according to claim 17, wherein the arithmetic unit performs the calculating and determining steps for a plurality of patients in parallel.

20. A medical-diagnostic blood analyzer, comprising:
at least one measuring sensor for measuring at least one physiological blood parameter of the samples,
an arithmetic unit including instructions therein causing the analyzer to:
receive measuring signals of the at least one measuring sensor taken from a patient at discrete measuring times,
process the measuring signals to determine measured values of the at least one physiological blood parameter;
determining a desired next measuring time;
determining whether the desired next measuring time falls within a measurement exclusion time window;
if the desired next measuring time falls within a measurement exclusion time window, providing for adjustment of one of the desired measuring time and the measurement exclusion time window such that the desired measuring time does not fall within the measurement exclusion time window;
defining the desired next measuring time as the recommended measuring time once the desired measuring time does not fall within the measurement exclusion time window; and
calculate a recommendation for a dosage of at least one active ingredient to be administered to the patient until a next measuring time on the basis of a dosage proposal algorithm incorporating the at least one measured physiological blood parameter, whereby at least one physiological parameter of the patient is adjusted to a target range.
